(19) United States
(12) Patent Application Publication
(10) Pub. No.: US 2025/0017755 A1
(43) Pub. Date: Jan. 16, 2025

(54) Flexibly Patterned Foot Orthoses for Improved Sensory Feedback

(71) Applicant: Thomas Smoot, JR., New York, NY (US)

(72) Inventor: Thomas Smoot, JR., New York, NY (US)

(21) Appl. No.: 18/220,199

(22) Filed: Jul. 10, 2023

(51) Int. Cl. A61F 5/14; A43B 17/14

(52) U.S. Cl. CPC A61F 5/14; A43B 17/14

(57) ABSTRACT

The present invention relates to an innovative insole design that enhances proprioceptive feedback in footwear. The invention utilizes a flexibly patterned mesh, made of thermoplastic polyurethane (TPU) and manufactured using additive manufacturing techniques, to create a three-dimensional lattice structure with interconnected cells. This unique design enables the novel mesh to compress in three dimensions, generating a distinctive pinching sensation on the skin of the foot when pressure is applied. The tactile interaction stimulates the nerves in the skin, enhancing proprioceptive feedback. This mesh can be incorporated into various orthotics, such as arch cookies and heel cups, and used in a wide range of shoes. It accommodates individuals with different foot conditions and improves balance, coordination, and gait while reducing the risk of falls. The flexibly patterned orthoses represent an innovative advancement in enhancing proprioception, offering a thinner, more flexible, and durable alternative to traditional insoles.

### BACKGROUND OF THE INVENTION

Proprioception is the ability to sense the position and movement of one's body parts. It is important for balance, coordination, and gait. Proprioception can be impaired by a variety of factors, including injury, disease, and aging.

There are a number of devices that can be used to improve proprioception. One type of device is an insole. Insoles can provide proprioceptive feedback by stimulating the nerves in the skin of the foot. This feedback can help to improve balance, coordination, and gait.

The present invention introduces a novel approach to enhance proprioception in orthotics through the utilization of a flexibly patterned mesh. This novel mesh, composed of thermoplastic polyurethane (TPU), employs additive manufacturing techniques to create a unique surface that interacts with the foot. By reducing solid TPU material to a uniform lattice structure, voids are formed, allowing for increased flexibility and compressibility in three dimensions. When pressure is applied during walking or other weight-bearing activities, the mesh compresses and produces a distinctive pinching sensation on the skin of the foot. This tactile interaction is believed to enhance proprioceptive feedback by stimulating the sensory nerves in the skin, thereby improving balance and stability.

This mesh has the potential to improve the quality of life for people with a variety of foot conditions, including injury, disease, and aging. The mesh forms multiple types of orthoses, that can also be used by people who need shock absorbing support to address specific, repetitive use injuries, in addition to proprioceptive feedback provided by contact with the mesh.

### SUMMARY OF THE INVENTION

A flexible foot orthosis is a new type of orthosis that is made from a flexible mesh material. The mesh material is designed to provide support and flexibility, while also being lightweight and breathable. The mesh is also resistant to water and dirt, making it ideal for use in a variety of environments.

### BRIEF DESCRIPTION OF DRAWINGS

FIG. 1 shows a top plan view of footwear inserts.

FIG. 2 shows a bottom plan view of footwear inserts.

FIG. 3 shows a top perspective of the 3d printed, ringed mesh in the form of an insole.

FIG. 4 is a top perspective view of FIG. 3 illustrating the uniform lattice of the mesh structure in greater detail.

FIG. 5 is a bottom plan view of FIG. 2 illustrating the ringed edge of the mesh in greater detail.

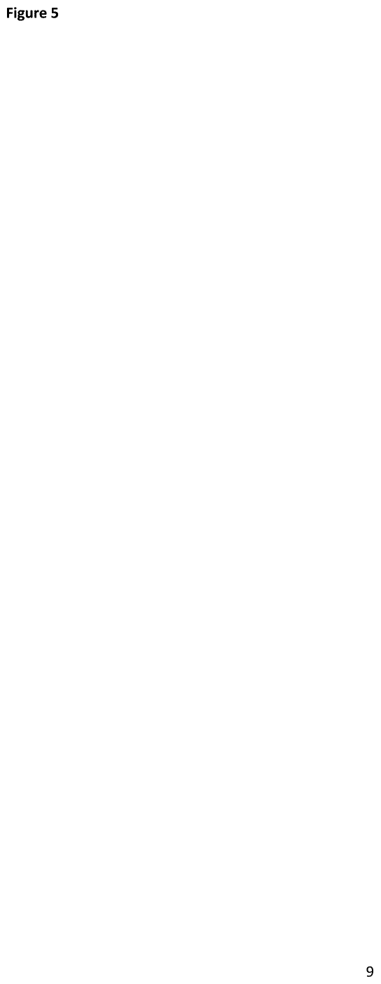

FIG. 6 is a top perspective view of FIG. 3 illustrating the uniform lattice of the mesh structure in greater detail.

In these figures, the broken lines show environmental structure and are for illustrative purposes only.

### DETAILED DESCRIPTION OF THE INVENTION

The present utility patent application is related to a pending design patent application, assigned application Ser. No. 29/882,469, filed on Jan. 14, 2023, which covers the ornamental design of 3D Printed Mesh With A Ringed Edge For Footwear Inserts.

The present invention introduces an innovative orthotic design that enhances proprioceptive feedback through the utilization of a flexibly patterned mesh. This mesh is formed using solid TPU as a raw material in the layer-by-layer construction of an additive manufacturing process. The first step in this process is shaping the solid TPU through the use of computer aided modeling software that reduces the TPU material used by removing density of a solid model to a lattice structure with uniform voids.

The density of material, and subsequently the mesh created from this process, is optimized to account for the force expected by the average human adult. This manufacturing technique allows for the creation of a unique three-dimensional structure, comprised of interconnected cells with uniform dimensions of less than three millimeters in diameter to absorb impact forces applied while completely compressing in three dimensions to provide tactile sensation. However, it should be understood that slight deviations from these dimensions may be necessary to accommodate variables specific to TPU manufacturers, such as differences in shore hardness. These adjustments ensure that the lattice structure retains its desired characteristics, including the ability to compress and provide the intended shock-absorbing and proprioceptive attributes.

The mesh pattern is then applied to specific orthotic products that provide foot support based on medical necessity, such as an arch support for diagnoses related to plantar fasciitis and other foot arch issues, heel cups for diagnoses related to heel pain and ankle stability, or full-length insoles that increase skin contact to improve proprioception across the entire foot. Each orthotic device is produced through a standard additive manufacturing process that allows for more detailed orthoses than traditional manufacturing, such as injection molding.

By employing these additive manufacturing techniques, the mesh can be activated when pressure is applied, such as during walking or weight-bearing activities. The flexible and durable mesh undergoes controlled compression, resulting in a distinct pinching sensation on the skin of the foot. This tactile interaction serves to stimulate the nerves in the skin, contributing to the improvement of proprioceptive feedback.

In prior art cases, various materials such as TPU, Ethylene Vinyl Acetate (EVA), and other flexible materials have been utilized in two-dimensional patterns, such as honeycomb or diamond patterns, extruded from the two-dimensional pattern with the aim of reducing impact on the foot. Much of the prior art shows some effectiveness in providing cushioning and shock absorption by utilizing patterns and meshes that vary in density based on regions of highest impact. They generally lack the ability to specifically stimulate the skin based on localized force application.

Prior art that is successful at providing increased proprioception are typically solid materials that employed a textured surface such as a ‘plurality’ of protrusions or flexible members to enhance proprioception. However, the configuration and arrangement of these protrusions often lack the ability to utilize the compressive force applied as a mechanical action that can grip or pinch in the same small scale as the present invention, and as a result many fail to provide highly targeted stimulation.

The present invention significantly improves upon these prior art approaches by introducing a novel method that combines additive manufacturing techniques with a lattice mesh structure. By leveraging the benefits of additive manufacturing, the mesh incorporates uniform, flexible cells arranged in a highly specific lattice pattern, designed to stimulate the skin of the foot based on localized force application. The lattice structure provides the ability to precisely control the distribution and interaction of the cells, resulting in enhanced proprioceptive feedback.

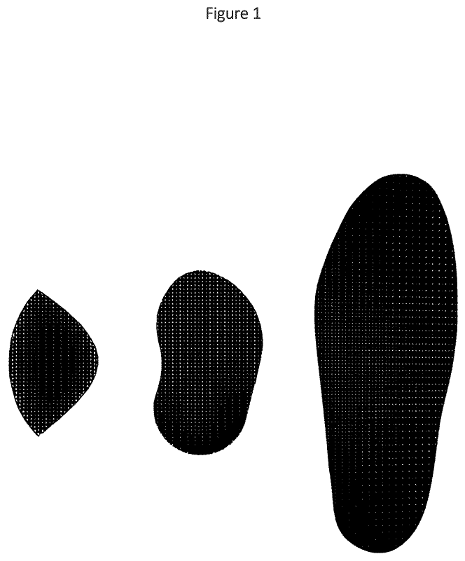

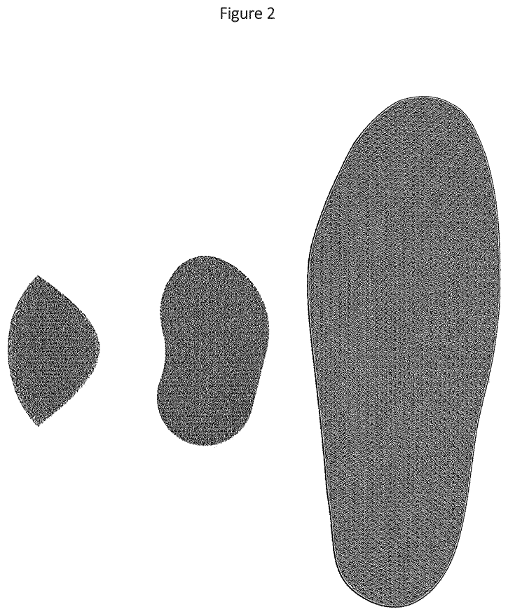

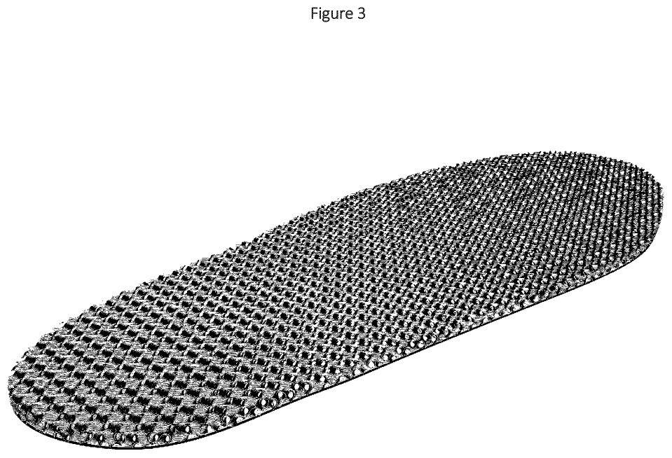

FIGS. 1 and 2 show the mesh employed in the form of specific orthoses to support multiple medical conditions, while FIG. 3 shows the top perspective view of a single insole.

A ring at the base of each orthosis can be seen in figures two, three, and five. The style of the ring shown is ornamental. However, incorporating this ring does serve a basic function, as it improves the manufacturing process and the durability of the orthosis. The ring is strategically designed to enhance adhesion during the additive manufacturing process by providing a stable border when the first layer of TPU is applied to the build surface during additive manufacturing. Furthermore, after production, the ring acts as a protective border, enhancing the overall durability and longevity of the printed orthosis.

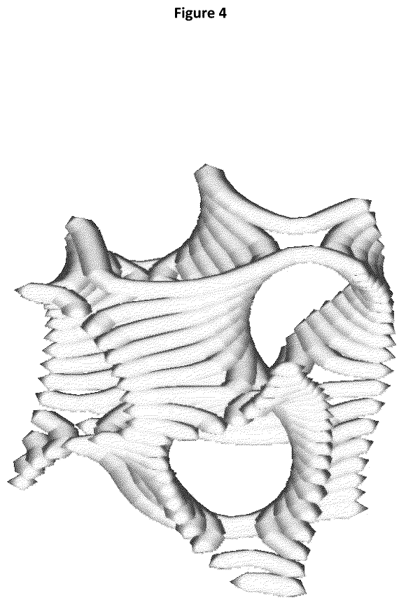

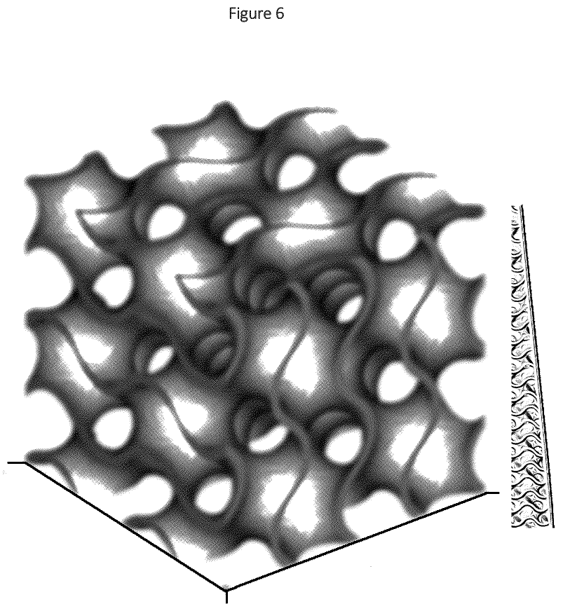

FIGS. 4 and 6 depict cross-sectional views of the insole, showcasing a cube that has been removed from the center of the insole. These cross sections provide a comprehensive visualization of the internal structure of the insole, including the arrangement of the flexible cells and the lattice pattern that gives rise to the voids. FIG. 6 accurately represents the uniform cells, while FIG. 4 demonstrates the formation of the voids from the cells depicted in FIG. 6, as facilitated by the layer-by-layer application during the additive manufacturing process. The dimensions of the cube effectively illustrate the size and configuration of the cells within the insole, providing insight into their structural characteristics in relation to localized forces.

The introduction of these flexibly patterned orthoses represents a significant advancement in enhancing balance, coordination, and gait, while mitigating the risk of falls. Its innovative design surpasses traditional insoles in terms of thinness, flexibility, and durability. By leveraging the benefits of additive manufacturing and its unique tactile characteristics, the underlying mesh opens new possibilities for improving proprioception in footwear applications. Its versatility allows for integration across various shoe types, catering to individuals with different foot conditions seeking enhanced proprioceptive feedback in addition to overall shock absorbing performance.

1. A foot orthosis comprising: a flexibly patterned mesh having a uniform lattice structure; and a simple exterior support member for supporting the flexibly patterned mesh in a shoe.

2. The foot orthosis of claim 1, wherein the flexibly patterned mesh is made of thermoplastic polyurethane.

3. The foot orthosis of claim 1, wherein the flexibly patterned mesh is configured to compress in three dimensions when force is applied to it.

4. The foot orthosis of claim 1, wherein the voids within the flexibly patterned mesh have uniform dimensions optimized to utilize local force applied to completely compress locally in three dimensions.

5. The foot orthosis of claim 1, wherein the flexibly patterned mesh provides proprioceptive feedback by stimulating the nerves in the skin of the foot.

6. The foot orthosis of claim 1, wherein the foot orthosis is used to improve balance, coordination, and gait.

7. The foot orthosis of claim 1, wherein the foot orthosis is used to reduce the risk of falls.
